(19) United States
(12) Patent Application Publication
(10) Pub. No.: US 2004/0193075 A1
(43) Pub. Date: Sep. 30, 2004

(54) System for foot assessment including a device and method

(76) Inventor: Michael Martindale, Portland, OR (US)

Correspondence Address: BRADLEY M GANZ, PC, P O BOX 10105, PORTLAND, OR, 97296

(21) Appl. No.: 10/808,878

(22) Filed: Mar. 24, 2004

Related U.S. Application Data
(60) Provisional application No. 60/457,869, filed on Mar. 25, 2003.
(60) Provisional application No. 60/463,661, filed on Apr. 16, 2003.

(51) Int. Cl. A61B 5/103

(52) U.S. Cl. 600/592; 33/515

(57) ABSTRACT

The present invention is a device and method of assessing the amount of pronation of a foot. The device includes a marker applied to the talar-head region of the foot. As the foot displaces from a first, subtalar-joint-neutral position to a second, relaxed position, corresponding movement of the marker is observed. The amount of movement in the marker relates to the amount of pronation of the foot. An optional template aids observation of this movement. The method assesses pronation by observing the talar-head displace as the foot transitions from a subtalar-joint-neutral position to a relaxed position. Optionally, the method includes providing a marker and a template to aid observation.

### PRIORITY CLAIM

Priority is claimed to co-pending provisional patent application No. 60/457,869 entitled SYSTEM FOR FOOT ASSESSMENT INCLUDING A DEVICE AND METHOD filed by the inventor on 25 Mar. 2003 and co-pending provisional patent application No. 60/463,661 entitled IMPROVED SYSTEM FOR FOOT ASSESSMENT INCLUDING A DEVICE AND A METHOD filed by the inventor on 16 Apr. 2003, the entire disclosures of which are hereby incorporated by reference and set forth in their entirety for all purposes.

### BACKGROUND OF THE INVENTION

This invention relates to foot assessment systems. Specifically, this invention relates to a device and a method to assess the amount of pronation of a foot.

Pronation is a complex motion in three dimensions and involves rotation among multiple axes of the foot, ankle, and leg. Normally, the foot adapts to the surface it lands upon, slightly rotates inward, and flattens the arch as the heel hits the ground. The foot's ability to pronate assists the body's mid-stance balance and is a part of a natural shock-absorbing system.

Pronation is tri-planar motion of the foot; it consists of eversion, abduction, and dorsiflexion at the subtalar joint. Eversion (and oppositely, inversion) occurs in the frontal plane: The foot everts when it twists outward and upward, rotating the plantar surface (or sole) away from the center. Abduction (and oppositely, adduction) occurs in the transverse plane: the foot abducts when it rotates laterally, away from center. Dorsiflexion (and oppositely, plantarflexion) occurs in the sagittal plane: the foot dorsiflexes when it moves upwards, toward the tibia. Oppositely, supination is outward rotation of the ankle and the outside border of the foot supports the body.

A normal amount of pronation and supination is beneficial; it is the body's way to absorb shock, create a more stable and rigid platform for push-off, and achieve dynamic balance. Excessive motion in either direction can be very problematic if not controlled and predisposes the lower extremity to injury. An excessive amount of pronation (over- pronation) can be problematic because the shifting causes increased stress on the inside, or medial aspect, of the foot. Over-pronation pulls on the stabilizing muscles in the lower leg (posterior tibialus). The body may compensate for over-pronation by excessive internal rotation of the lower extremity and shifting of the subtalar-joint axis and midtarsal joint axis medially, for example. This may result in injuries to the knee or Achilles tendon. Conversely, excessive supination stretches the stabilizing muscles on the outside of the lower leg (peroneals) and the ankle may roll over.

It is often necessary to correct foot-motion when a foot over pronates. The correction may greatly reduce the propensity for injury and improves ambulatory performance. An over-pronating foot can be corrected with remedial foot support that aligns the foot in a normal pronation-range. Orthotic-insoles, also called prescription foot orthotics, are one approach to properly align and support the foot. Orthotic insoles are custom-made inserts for shoes and are designed to correct various foot and lower body conditions. The manufacturing and materials vary based on patient needs, activities, and health factors. Another approach is to select motion-control shoes, or shoes with stabilizing features, that can correct the range of motion of an over-pronating foot.

To select the appropriate orthotic insole or motion-control shoe, the amount of pronation must be assessed. One prior-art assessment technique teaches videotaping the foot while running. Pronation is determined by viewing the motion of the rear foot, or movement of the calcaneus in the frontal plane. Assessing pronation based on rear-foot motion is inaccurate. For example, a foot can exhibit a small amount of calcaneal eversion yet have severe over-pronation. Alternatively, the amount of calcaneal eversion can be limited in the frontal plane but severe rotation may occur in the transverse plane.

Another prior-art attempt to assess pronation requires a visual inspection of the worn tread on a pair of shoes. This is highly subjective, as it depends on the skill of the observer.

Another prior-art assessment method quantifies the lowering of the longitudinal-arch profile. This method is unsatisfactory: for example, a foot can maintain a high arch-profile but exhibit severe transverse-plane and frontal-plane motion at the subtalar joint and midtarsal joint.

Each of the prior-art methods does not efficiently and accurately assess pronation. The prior-art systems are cumbersome, complicated, and require skill to use. The prior-art systems are also imprecise and subjective. A more accurate and simpler approach of assessing pronation is needed.

### SUMMARY OF THE INVENTION

This novel invention presents a device and method for assessing the amount of pronation in a foot. The device and method are easy to use, do not require specialized skill, and greatly reduce subjectivity in assessing pronation.

Contrary to findings of the prior-art, the amount of pronation (including over-pronation) is independent from arch height, rear-foot to fore-foot alignment, or the position of the calcaneus relative to the lower leg. Also contrary to the teaching of the prior-art, assessing pronation does not require precise measurements of the foot's movement in the three anatomical planes or measurement of the rotation of the subtalar joint and midtarsal joint.

As subsequently explained in further detail, pronation of the foot may be directly related to corresponding movement of the talar-head. By observing the displacement of the talar-head as the foot rotates from a first, subtalar-joint-neutral position to a second, relaxed position, an amount of natural pronation may be assessed.

In one embodiment, the device includes a marker adapted for application to a talar-head region of the foot. The marker facilitates observation of the talar-head region of the foot as it displaces from a first position to a second position.

In another embodiment, the device includes a template having a first region adapted to represent a first amount of pronation (normal-range of pronation) in the second position and a second region representing a second amount of pronation (over-pronation) of the foot in the second position.

Another embodiment of the device combines the indicator and template. The indicator aids visual inspection of the foot in a first position and a second position, and the template aids initial alignment and may quantify relative displacement of the indicator. The template has a first region representing a first amount of pronation of the foot in the second position and a second region adapted to represent a second amount of pronation of the foot in the second position.

In one embodiment a method of assessing the amount of pronation in a foot includes observing the talar-head region of the foot in a subtalar-joint-neutral position and watching the talar-head region of the foot rotate from the subtalar-joint-neutral position to a relaxed position.

In an alternative embodiment, a method of assessing the amount of pronation in a foot includes providing a marker and attaching the marker to a talar-head region of the foot. The method also includes placing the foot in a first position, moving the foot to a second position, and observing the movement of the marker as the foot moves from the first position to the second position. The movement of the marker corresponds to the amount of pronation of the foot.

Another embodiment of a method of assessing the amount of pronation in a foot includes providing a template and aligning the foot in a subtalar-joint-neutral position. The method also includes relaxing the foot to a second position and observing the displacement of a talar-head region of the foot.

Another embodiment of the present invention includes a method of selecting footwear. The method includes placing the foot in a first position, moving the foot to a second position, observing the relative displacement of the indicator from the first position to the second positions, and selecting footwear based on the relative displacement of the foot, which corresponds to an amount of pronation.

The foregoing and other objects and aspects of the present invention are explained in detail in the specification set forth below.

### BRIEF DESCRIPTION OF THE DRAWINGS

FIGS. 1A-1C show a human foot and associated anatomical planes.

FIG. 2 shows the bones of the foot of FIG. 1.

FIG. 3 shows several reference lines associated with the foot of FIG. 1.

FIG. 4 is a schematic representation of the foot in a first position in relation to one embodiment of the present invention.

FIG. 5 is a profile of the image of FIG. 4.

FIG. 6 is a schematic representation the foot of FIG. 4 in a second position.

FIG. 7 shows another embodiment of the present invention

### DESCRIPTION OF THE INVENTION

The present invention will now be described more fully hereinafter with reference to the accompanying figures, in which preferred embodiments of the invention are shown. This invention may, however, be embodied in many different forms and should not be construed as limited to the embodiments set forth herein. Like numbers refer to like elements throughout. In the figures, certain components, features or layers may be exaggerated for clarity.

This invention overcomes problems of prior-art pronation assessment systems. Specifically, it is more accurate, is simpler to use, does not require specialized skill, and greatly reduces subjectivity.

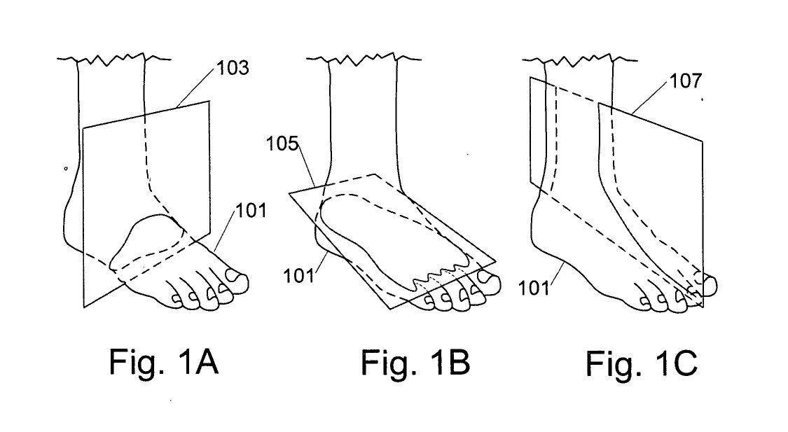

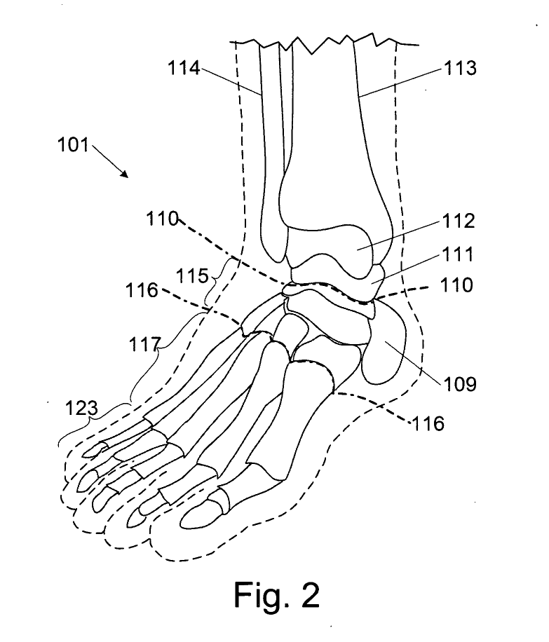

FIGS. 1A, 1B, and 1C show three anatomical planes with respect to a human foot 101: the frontal plane 103, the transverse plane 105, and the sagittal plane 107. The frontal plane divides the foot front-to-back. The transverse plane runs through the heel and divides the foot top-to-bottom. The sagittal plane divides the foot left-to-right. FIG. 2 shows the bones of the foot of FIG. 1. The foot 1 includes seven tarsal bones 115, five metatarsal bones 117, the phalanges 123, the calcaneus 109, and the talus 111. Also referenced are the talar-head 112, tibia 113 and the fibula 114. FIG. 2 also shows various reference lines with respect to the foot 101 of FIG. 1 including lines representing the transmalleolar axis (TMA) 119, subtalar joint 110, and midtarsal joint 116.

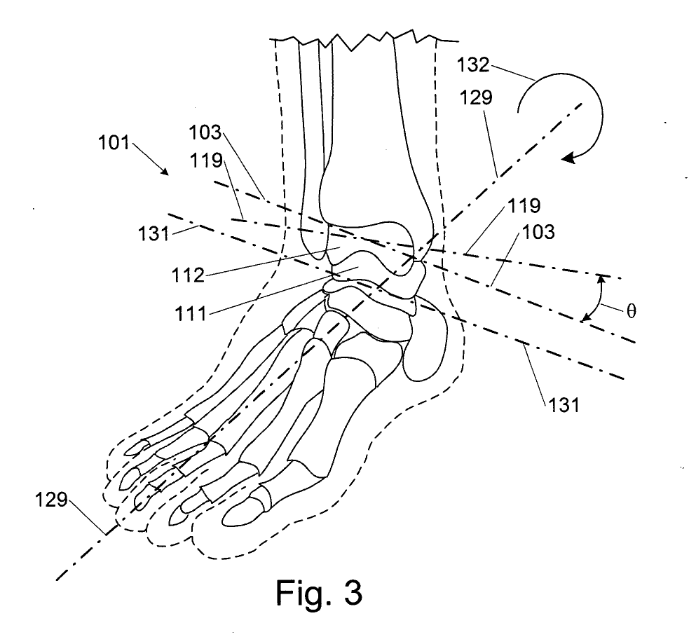

This invention is based the observation that an over-pronated foot has an abnormally medially positioned talar-head 112. The talar-head is internally rotated in relation to the calcaneus 109. Thus, the more medially deviated the subtalar-joint axis 131 (shown in FIG. 3), the greater the magnitude of pronation. The amount of pronation can be observed when the foot rotates from a subtalar-joint-neutral position to a relaxed position. This pronation-based rotation is translated to movement of the talus 111 because both the subtalar-joint axis 131 and the midtarsal-joint-oblique-axis 129 pass through the talus 111.

The present invention exploits the discovery that pronation may be re-defined as medial deviation 130 of the subtalar-joint axis 131, rotation 132 about the midtarsal-joint-oblique-axis 129, and adduction and plantarflexion of the talus 111. This motion manifests itself as movement of the talar-head 112. Accordingly, the present invention includes a device 1 that facilitates observation of motion of the talar-head 112 as the foot 101 displaces from the subtalar-joint-neutral position to the relaxed position.

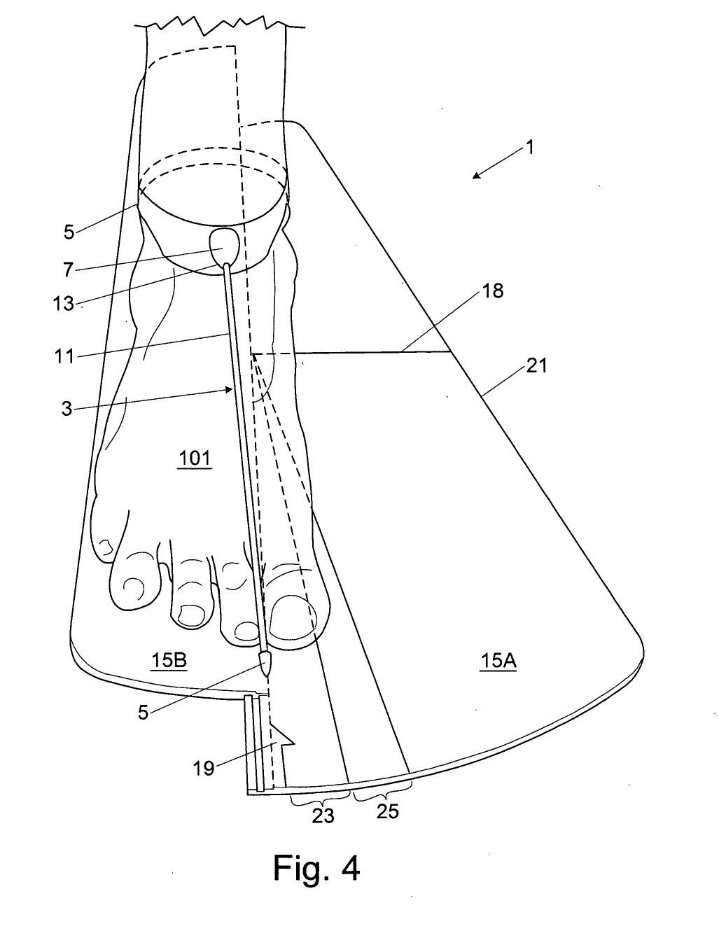

FIGS. 4-6 show a possible embodiment of the present invention. The device includes a marker, for example, indicator 3. The elongated indicator 3 has a relatively long shaft 11 with a distally located tip 9. At an end opposite the tip 9, the base 13 of the shaft is coupled to a mounting surface 5 having means for attaching to the foot 101. The means for attaching to the foot may be straps, tape, or self-adhesive pad, for example. The shaft 11, firmly secured to the talar-head 112, amplifies rotation of the foot as it transitions from a first position to a second position.

The device may further include an optional template 21. The template adapts to align the foot in an initial or first position. For example, the template 21 includes an alignment guide, such as reference line 19. When the foot is in the sub-talar joint neutral position (initial position) and the indicator 3 is properly adjusted and secured, the longitudinal axis of the shaft 11 will coincide with the reference line 19.

The template 21 may include two plates (15A and 15B), which are adjustable with respect to each other. Each plate 15A and 15B may include an ankle-joint reference line 18, which corresponds with the foot's transmalleolar axis 119 when the foot 101 is properly aligned in the subtalar-joint-neutral position. Typically, the TMA 119 is inclined about 20 to about 23 degrees in lateral rotation (indicated by “θ” in FIG. 3) to the frontal plane when viewed from a perspective normal to the transverse plane 105. Aligning the anklebone with ankle-joint reference line 18 also may align the foot 101 in the first, subtalar-joint-neutral position.

The template 21 also receives the foot in second, relaxed position. One enhancement to the template 21 may include a first pronation zone 23, indicating a normal range of pronation, and a second zone 25, indicating over-pronation.

In another embodiment, the device 1 may include only a template 21. In another embodiment, the device 1 may include only a marker. In fact, any combination of template, marker, both, or neither will assist observation of the talar head to varying degrees.

The foot assessment device 1 may be made from a variety of materials such as paper, plastics, wood, lightweight metals, or various alloys. For example, a paper template might be mailed to a user for home-assessment. In another embodiment, the foot alignment template 21 or relevant lines or points thereon is drawn or marked on the floor or other object that the foot stands upon. The device may be provided to a point-of-sale location to facilitate the selection of footwear.

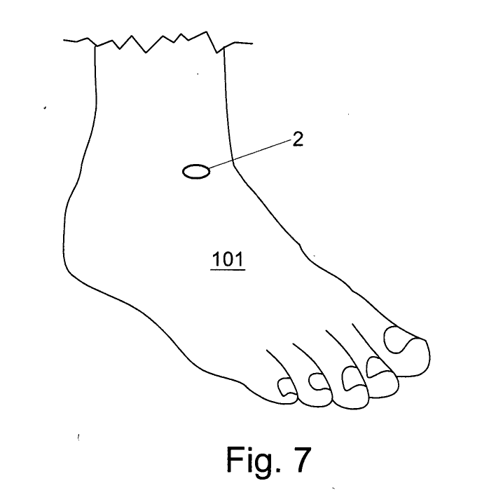

The marker may be anything that facilitates observation of movement of the talar-head region 112 of the foot as it moves from the first position to the second position. For example, as shown in FIG. 7, the marker 2 could be an ink spot.

In another possible embodiment, the device 1 may comprise a marker 2 made from a reflective material. A light source, such as an IR or laser light, may be reflected off the marker 2. As the talar-head rotates from the subtalar-joint-neutral position to the relaxed position, a sensor detects the amount of displacement. A processor calculates the displacement and relates the displacement to an amount of pronation and sends this information to an output device. Optionally, the output device selects a recommended range of footwear or orthotic insert from a database, which may be internal to the device, or remote and connected via a data-network, such as the Internet, for example.

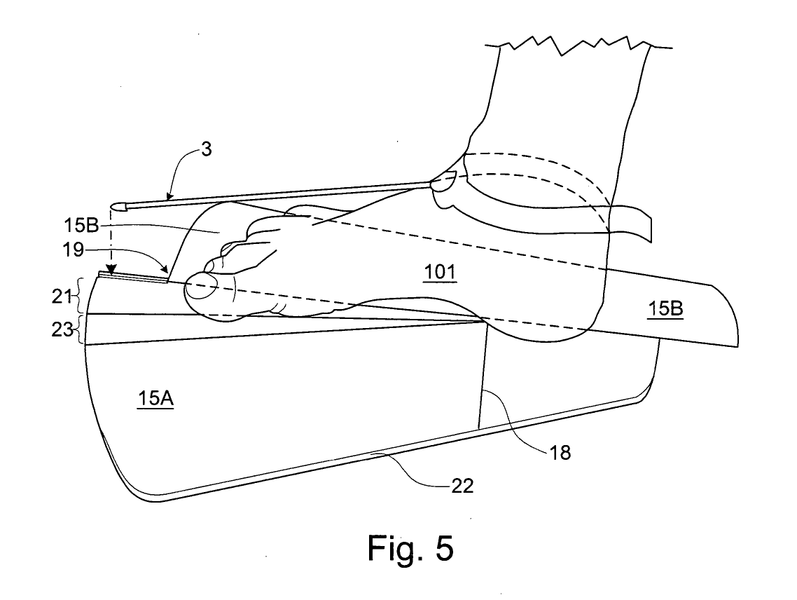

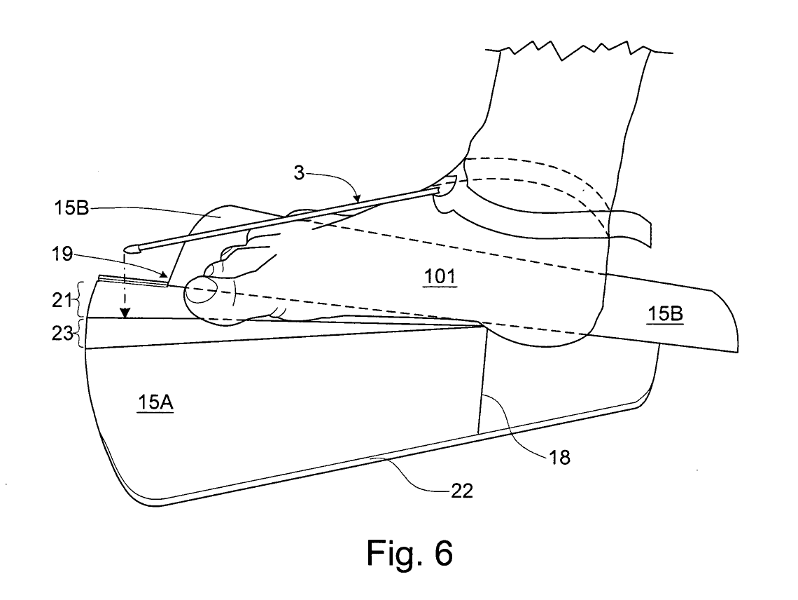

In another embodiment of the present invention includes a method to determine the relative amount of pronation. The method may be used independent of any particular device and may be based solely on observation of relative movement of the foot. The method includes the steps of placing a subject foot 101 in the subtalar-joint-neutral position (shown in FIG. 5, for example). An observer observes the location of the foot 101 in this first position. Next, while watching the talar-head region 112, the foot rotates to a second, relaxed position (shown in FIG. 6, for example). The amount of rotation of the talus 111 at the talar-head 112 corresponds to the amount of pronation.

Optionally, the method according to the present invention may incorporate the device 1 as described herein. A template 21 is placed under the foot 101. A marker 2 or an indicator 3 is attached to the foot over the region near the talar-head 112. Then, the foot 101 is placed in the subtalar-joint-neutral position. The indicator 3 is secured to the foot and adjusted so that the longitudinal axis of the indicator coincides with a reference line 19 when viewed from a normal perspective to the template 21. The observer observes an initial position 15 of the indicator 3. Next, the foot 101 rotates to the relaxed position. The observer observes the second position of the indicator 3 in relation to the template 21. The relative movement from the indicator's first position 15 to the second position 17 correlates to the amount of pronation. The template 21 may include aids to assess pronation including pronation zones: a first zone 23 indicates a range of normal-pronation, a second zone 25 indicates over-pronation. Thus, when the foot 101 is in the relaxed position and the indicator 3 is viewed generally normal to the template 21, the tip 9 overlaps one of the regions on the template 21.

Another embodiment of the present invention includes a method of selecting footwear. This method includes placing the foot 101 in a first position, moving the foot to a second position, observing the relative displacement of the talar-head 112—the displacement corresponding to an amount of pronation of the foot, and selecting footwear based on the amount of pronation. For example, an over-pronating foot may benefit from a stability or motion-control running shoe.

Another possible embodiment may include a database, which may contain a classification of shoes, orthotic inserts, or both. The database may be organized in any logical manner, such as, brand, activity type (running, walking, hiking, cross-training, standing, or suitable for individual recovering from medical procedures, for example), model, pronation-correcting type, or other useful categories, for example. The database may be accessed via the Internet or an intranet, it may reside on a personal computer, or it may be a look-up table that is conveniently located at a point-of-sale, for example.

The foregoing is illustrative of the present invention and is not to be construed as limiting. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

What is claimed:

1. A foot assessment device comprising:
a marker adapted for application to a talar-head region of the foot and adapted to aid visual inspection of the foot in a first position and a second position.

2. The device of claim 1 wherein the marker comprises an elongated shaft.

3. The device of claim 1 further comprising:
a template adapted to align the foot in the first position.

4. The device of claim 1 further comprising:
a template adapted to receive the foot in the second position.

5. The device of claim 1 further comprising:
a template adapted to align the foot in the first position and the template further adapted to receive the foot in the second position.

6. The template of claim 5 further comprising:
a first region adapted to represent a first amount of pronation of the foot in the second position; and
a second region adapted to represent a second amount of pronation of the foot in the second position.

7. A device for assessing pronation of the foot, the device comprising:
a template comprising a first region adapted to represent a first amount of pronation of the foot in the second position, and a second region adapted to represent a second amount of pronation of the foot in the second position.

8. The template of claim 7 further comprising an alignment guide adapted to align the foot in the first position.

9. The template of claim 8 wherein the alignment guide is adapted to align the foot in a subtalar-joint-neutral position.

10. A device for assessing pronation in the foot, the device comprising:
an elongated indicator adapted for application to a talar-head region of the foot and adapted to aid visual inspection of the foot in a first position and a second position;
a template adapted to align the foot in the first position and the template further adapted to receive the foot in the second position; and
the template further comprising a first region adapted to represent a first amount of pronation of the foot in the second position, and a second region adapted to represent a second amount of pronation of the foot in the second position.

11. A method of assessing the amount of pronation in a foot, the method comprising:
observing the talar-head region of the foot in a subtalar-joint-neutral position; and
assessing the amount of pronation by watching the talar-head region of the foot rotate from the subtalar-joint-neutral position to a relaxed position.

12. A method of assessing the amount of pronation in a foot, the method comprising:
providing a marker;
attaching the marker to a talar-head region of the foot;
placing the foot in a first position;
moving the foot in a second position; and
observing the movement of the marker as the foot moves from the first position to the second position, and the movement corresponding to the amount of pronation of the foot.

13. A method of assessing the amount of pronation in a foot, the method comprising:
providing a template;
aligning the foot in a subtalar-joint-neutral position;
relaxing the foot to a second position; and
observing the displacement of a talar-head region of the foot.

14. A method of selecting footwear comprising:
placing the foot in a first position;
moving the foot to a second position;
observing the relative displacement of the foot from the first position to the second position, the displacement corresponding to an amount of pronation of the foot; and
selecting footwear based on the amount of pronation.

15. The method of claim 13 wherein placing the foot in a first position further includes:
placing the foot in a subtalar joint-neutral position.

16. The method of claim 13 wherein moving the foot to a second position further includes moving the foot to a relaxed position.

17. The method 13 of claim further comprising providing an elongated indicator.

18. The method of claim 13 further comprising providing a template.

19. The method of claim 14, where the amount of pronation is matched to one or more items of footwear stored in an electronic database.

20. The method of claim 19 where the amount of pronation is match to the database via an electronic data network.

21. A device for assessing pronation in the foot, the device comprising:
a marker including a reflective material and adapted for application to a talar-head region of the foot;
a light source to reflect light off the marker to measure displacement of the talar head as it moves from a first position to a second position; and
a processor for calculating the displacement and relating the displacement to an amount of pronation.

22. The device of claim 21 further comprising an output device for displaying the amount of pronation.

23. The device of claim 21 further comprising a database for storing a selection of footwear related to the amount of pronation of the foot.
